(19) United States
(12) Patent Application Publication
(10) Pub. No.: US 2017/0120002 A1
(43) Pub. Date: May 4, 2017

(54) REMOTE CONTROL SYSTEM AND METHOD FOR USE IN INTRALUMINALLY OR INTRAVASCULARLY LOCATED OPERATIONS

(71) Applicant: TARYAG MEDICAL LTD., Caesarea Industrial (North) (IL)

(72) Inventors: Swi BARAK, Caesarea (IL); Aharon COHEN, Ramot Hashavim (IL)

(21) Appl. No.: 15/318,700

(22) PCT Filed: Jun. 25, 2015
(86) PCT No.: PCT/IL2015/050659

§ 371 Date: Dec. 14, 2016

(30) Foreign Application Priority Data
Jun. 26, 2014 (IL) 233423

(51) Int. Cl. A61M 25/01; A61B 17/3207; A61M 1/00; A61B 17/34; A61M 39/06

(52) U.S. Cl. CPC A61M 25/0113; A61B 17/3476; A61M 39/06; A61M 1/0023; A61B 17/3207; A61B 2017/00212

(57) ABSTRACT

In a remote control system and method for use in intraluminally or intravascularly located operations, an invasive element to which a remote control unit (RCU) is attached is introducible into a lumen. An elongated tube, to which an activation device is affixed near a distal end, is feedable into the lumen via the invasive element. The RCU is positionable within transmission range of an actuator for the activation device. An operator manipulating the tube with one hand to receive a tactile sensation and engaging the RCU with another hand simultaneously views the tube distal end on a monitor, and depresses an RCU button to actuate the activation device after determining that a body portion is in need of a corrective action. In one embodiment, the RCU comprises a shell for securing the invasive element, facilitating application of a reactive force to the shell by a finger during an operation.

### FIELD OF THE INVENTION

The present invention relates to the field of medical equipment. More particularly, the invention relates to a remote control system for use in intraluminally or intravascularly located operations.

### BACKGROUND OF THE INVENTION

The introduction of a catheter within a bodily part allows various procedures to be performed including, but not limited to, drainage of accumulated fluid such as from the urinary bladder or from an abscess, administration of fluids or medication, and angioplasty. Success of a procedure is contingent upon accurate guidance of the catheter to the bodily part and timely activation of a component that is adapted to effect the procedure. An actuator for the component to be activated is generally positioned near the proximal end of the catheter.

The physician performing the procedure manually guides the catheter through an introducer sheath and over a guidewire, and the instantaneous location of the catheter is able to be visualized by several techniques such as fluoroscopy and ultrasound imaging. However, when it is desired to perform the procedure, the physician has to release his hands from the catheter or the introducer sheath in order to operate the actuator or to request assistance from a practitioner. Alternatively, the physician loses eye contact with the visualized imaging screen. Consequently the tactile sensation received from the catheter by the physician during the course of a procedure becomes impaired, and the distal tip of the catheter can no longer be guided or visualized. A subsequent corrective action has to be taken in order to reacquire the required control of the catheter. The valuable time of the physician manipulating a prior art catheter control system is therefore not effectively utilized, at times leading to a failure in timely performing a desired procedure.

It is an object of the present invention to provide a catheter based remote control system integrated with the introducer sheath, by which a physician can manipulate a catheter with one hand and operate a catheter carried activation device with the other hand.

Other objects and advantages of the invention will become apparent as the description proceeds.

### SUMMARY OF THE INVENTION

The present invention provides a catheter based remote control system, comprising an activation device located near or at a distal end of a catheter, for performing a desired intraluminally or intravascularly located operation, an actuator for said activation device near or at a proximal end of said catheter, and a remote control unit (RCU) positioned within transmission range of said actuator, wherein said RCU has a communication device for transmitting an activation signal for initiating operation of said activation device to said actuator.

The RCU is compact and user friendly such that the activation signal is transmittable to the actuator in response to interaction with the RCU by no more than three fingers.

The RCU is preferably attached to an invasive introducer device comprising a percutaneously insertable tube, such as an introducer sheath or a trocar, through which the catheter is introducible into a bodily part

In one aspect, the RCU is configured by a hollow shell for receiving the introducer sheath within its interior. A printed circuit board (PCB) for providing electronic capabilities of the RCU is embedded in a wall of the shell. An activation switch for initiating operation of the actuator is connected to the PCB and protrudes from the shell.

In one aspect, a safety switch for preventing inadvertent initiation of the actuator is connected to the PCB and protrudes from the shell, initiation of the actuator being enabled only if said safety switch is depressed within a predetermined time after the activation switch has been depressed.

In one aspect, the control system further comprises a visual or audible indicator for indicating actuator initiation.

The actuator is powered by an AC or DC current source.

In one aspect, the activation signal is wirelessly transmittable, and may be encoded. The activation signal may be an audio frequency signal, an infrared signal or a radio frequency signal, such as a unidirectional or bidirectional radio frequency signal in the ISM frequency bands.

In one aspect, the RCU comprises a microprocessor for interfacing with a networking protocol stack in order to generate the activation signal.

In one aspect, the RCU is a passive tag which is operable to respond to an interrogating signal generated by the activation device.

In one aspect, the actuator is a mechanical or electrical member activated by the RCU, such as a Motor and Pump Unit (MPU) for draining intraluminally accumulated liquid by applying subatmospheric pressure.

In one aspect, the activation device is an atherectomy device for removing atheromatous material from the walls of a blood vessel.

In one aspect, the RCU is a ring that is wearable on a finger or a hand portion of a physician performing a catheterization procedure.

In one aspect, the RCU and actuator are spaced by a distance ranging from 1 to 2000 cm that is variable during the course of the operation.

The present invention is also directed to a method for remotely controlling an activation device, comprising the steps of introducing a tubular sheath into a lumen of a body, wherein a remote control device is attached to a portion of said sheath externally to said body; feeding a flexible and elongated tube into said lumen via said sheath to initiate an intraluminal operation, wherein an activation device is affixed to a distal end of said tube; by an operator, while displacing said tube within said lumen with one hand to receive a tactile sensation which is indicative of a degree of body related resistance to displacement of said tube and engaging said remote control device with another hand, simultaneously viewing on a monitor a portion of said body corresponding to an instantaneous location of said tube distal end; and after determining that said body portion is in need of a corrective action in response to one or both of said tactile sensation and said viewing, depressing at least one button of said remote control device to actuate said activation device and to thereby perform said corrective action.

The operator continuously views on the monitor one or more portions of the body corresponding to an instantaneous location of the tube distal end within the body throughout the intraluminal operation.

### BRIEF DESCRIPTION OF THE DRAWINGS

In the drawings:

FIG. 1A is a schematic illustration of a catheter based remote control system, according to one embodiment of the present invention;

FIG. 1B is a schematic illustration of the remote control system of FIG. 1A after a catheter has been introduced into a bodily part;

FIG. 2A is a perspective view from the top of a remote control unit used in the system of FIG. 1A;

FIG. 2B is a perspective view from the bottom of the remote control unit of FIG. 2A;

FIG. 3 is a block diagram of a remote control system by which infrared signals are transmitted;

FIG. 4 is a block diagram of a remote control system by which unidirectional radio frequency signals are transmitted;

FIG. 5 is a block diagram of a remote control system by which bidirectional radio frequency signals are transmitted;

FIG. 6 is a block diagram of a remote control system by which high frequency radio frequency signals are transmitted;

FIG. 7 is a block diagram of a remote control system by which radio frequency signals are returned from a passive tag to an interrogator;

FIG. 8 is a block diagram of a remote control system by which audio frequency signals are transmitted;

FIG. 9 is a perspective view of another embodiment of a remote control unit; and

FIG. 10 is a flow diagram of a method for remotely controlling an activation device, according to one embodiment of the invention.

### DETAILED DESCRIPTION OF PREFERRED EMBODIMENTS

The remote control system of the present invention is adapted to control the functionality of a catheter carried activation device while the catheter is being displaced throughout the lumen of a patient's bodily part, and is manipulated by one hand of a physician while the other hand is guiding the catheter and the physician is visualizing the instantaneous location of the catheter within the body of a patient.

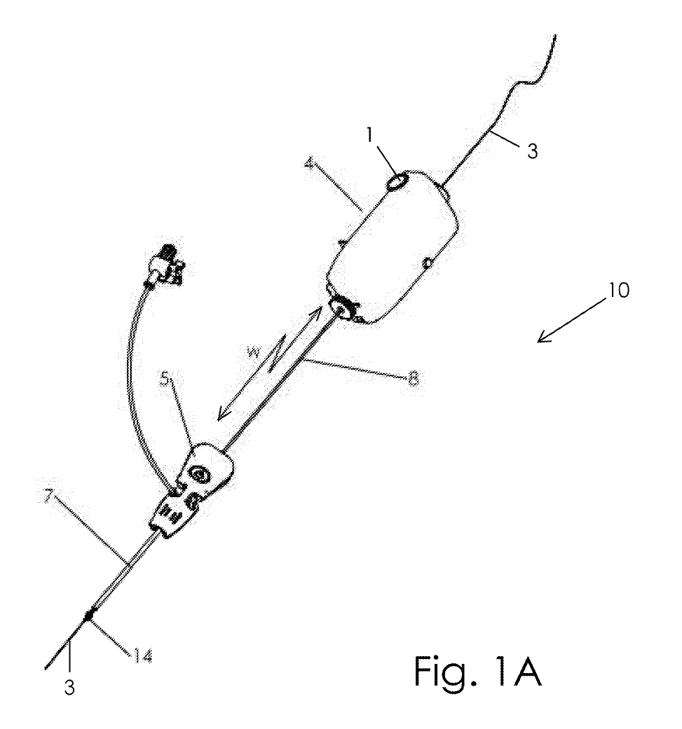

FIG. 1A schematically illustrates a catheter based remote control system, according to one embodiment of the present invention, which is generally indicated by numeral 10. A Remote Control Unit (RCU) 5 is attached, externally but close to the patient's body, to a tubular introducer sheath 7 through which a catheter 8 is introducible into a bodily part, and serves to communicate wirelessly by a signal W, whether unidirectionally or bidirectionally, with an actuator 4 to which the proximal end of the catheter is secured. Initiation of actuator 4 by signal W causes activation device 14 located near or at the distal end of catheter 8 to be operated. A hemostasis valve is generally provided at the proximal end of introducer sheath 7 to prevent leakage and contamination. Catheter 8 is guided along a previously positioned guidewire 3 extending within the interior of introducer sheath 7 and into a desired intraluminal or intravascular region. The distance between RCU 5 and actuator 4 is dependent upon the length of the catheter that has been introduced into the body of the subject, ranging from 1-2000 cm.

Actuator 4, which is generally positioned on a sterile surface during a catheterization procedure, is shown to have a rectangular configuration, but may be configured in any desired fashion, such as with a handle that is graspable by the physician or any other medical practitioner.

Remote control system 10 is operable in one of the following three modes, upon depressing a mode selector button 1 located on actuator 4: (1) the OFF mode whereby the electrical power source of activation device 14 is disconnected to prevent inadvertent activation, (2) the Remote Control mode for remotely initiating actuator 4 by RCU 5, and (3) the Direct Control mode for directly activating activation device 14 by manipulating an additional button (not shown) located on actuator 4. The controller of RCU 5 ensures that activation device 14 cannot be activated simultaneously in both the Remote Control and Direct Control modes.

FIG. 1B schematically illustrates remote control system 10 after catheter 8 has been introduced into bodily part 2.

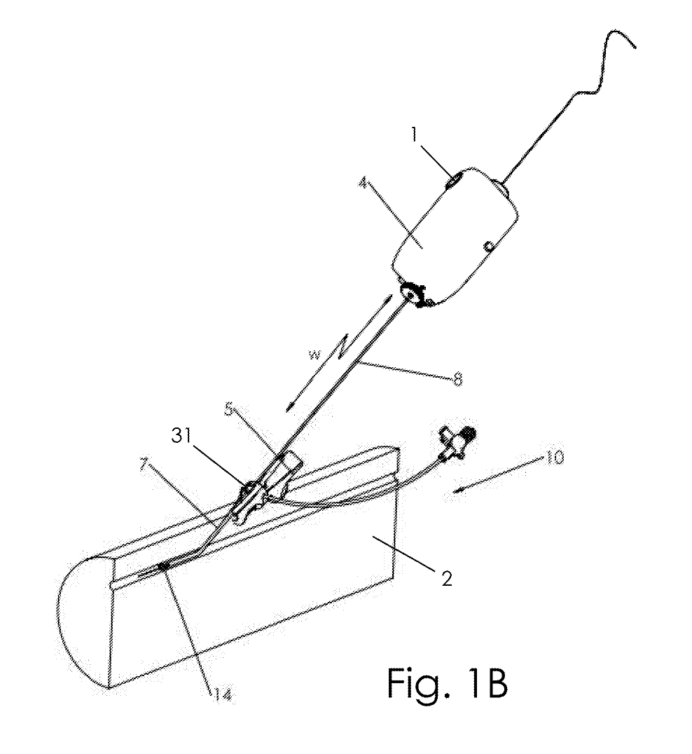

FIGS. 2A and 2B illustrate an exemplary configuration of RCU 5. RCU 5 comprises closed housing 10 in which is retained a printed circuit board (PCB) for providing its electronic capabilities, as will be described hereinafter, including a communication device for transmitting an activation signal to the actuator. Mounted within the upper surface of housing 10 is a finger depressable activation switch 28, which may be provided with an LED indicator for visual indication of when the activation switch is being depressed, or alternatively when the activation device is being operated.

If so desired, housing 10 may be provided with a safety switch, in order to prevent inadvertent initiation of the actuator. That is, the actuator will be initiated only if the safety switch will be depressed within a predetermined time, e.g. 0.5 sec, after activation switch 28 has been depressed.

RCU 5 also comprises an arcuate shell 6 that is configured to receive and secure introducer sheath 7 within its concave interior, including hemostasis valve 31 fixed to the proximal end of the introducer sheath, and through which catheter 8 extends until being secured by actuator 4, as shown in FIG. 1B. Side arm tube 17 of introducer sheath 7 through which medicament or blood is injectable is also connected to hemostasis valve 31, substantially perpendicularly to the longitudinal axis of introducer sheath 7. A stopcock 42 is fixed to the end of side arm tube 17. The upper surface of shell 6 is formed or otherwise provided with frictional enhancing elements 33, to assist a finger of the physician in properly engaging, and providing a reactive force to, shell 6 during a catheterization procedure.

A narrow and short spacer 9 extends from shell 6 to housing 10, to accommodate the provision of a small hook-like protrusion 38 extending from a circumferential edge 39 of shell 6 towards spacer 9. Although not shown in FIGS. 2A-B for purposes of clarity, side arm tube 17 of introducer sheath 7 is able to be fixated by hook-like protrusion 38.

It will be appreciated that RCU 5 may be configured in other ways in order to accommodate for example any other type of invasive introducer device or electronic components for communicating with the actuator.

In one embodiment, both the RCU and the actuator are mounted in a common housing from which the catheter proximally extends, in order to be manipulated during a catheterization procedure.

In the following description, the actuator is embodied by a Motor and Pump Unit (MPU), and particularly by a vacuum pump, for operating an activation device in the form of an aspiration tip intended for example to drain intraluminally accumulated liquid by applying subatmospheric pressure, but it will be appreciated that the invention similarly applies to an activation device for performing any other intraluminally or intravascularly located operation.

Non-limiting examples of a controllable actuator and activation device pair include a camera for a lens and shutter, an RF generator for a heating tip, an RF generator for a dissection or ablation tip in order to ablate tissue, a power source for a heating element, a positive pressure pump for a spray nozzle or for the inflation of a balloon, an AC or DC motor for a rotating tip in order to remove atheromatous material from the walls of a blood vessel, and a power source for a lamp.

A communication interface preferably, but not necessarily, extends from the actuator and through the catheter to the activation device. A medium needed by the activation device to perform a corrective action is transferred, or otherwise communicated, by means of the communication interface. The communication interface may be a mechanical connection for transmitting a force or energy to the activation device. Alternatively, the communication interface may be a channel through which a substance such as a medicament or a fluid is transferrable to the activation device, or by which information is optically or electronically transmittable.

The catheter may carry more than one activation device. A separate communication interface may be provided for each activation device. Alternatively, a single link extending from the actuator may be subdivided into a separate communication interface for each activation device.

The remote control system of the present invention is also applicable for use with a trocar during an intraluminal operation. A flexible tube carrying an activation tube is feedable within the trocar and into a bodily lumen. After the trocar is suitably introduced into the body of a patient, the RCU is attached to the trocar. The physician thus engages the RCU with one hand while the other hand is guiding the flexible tube and the physician is viewing the instantaneous location of the activation device within the patient's body.

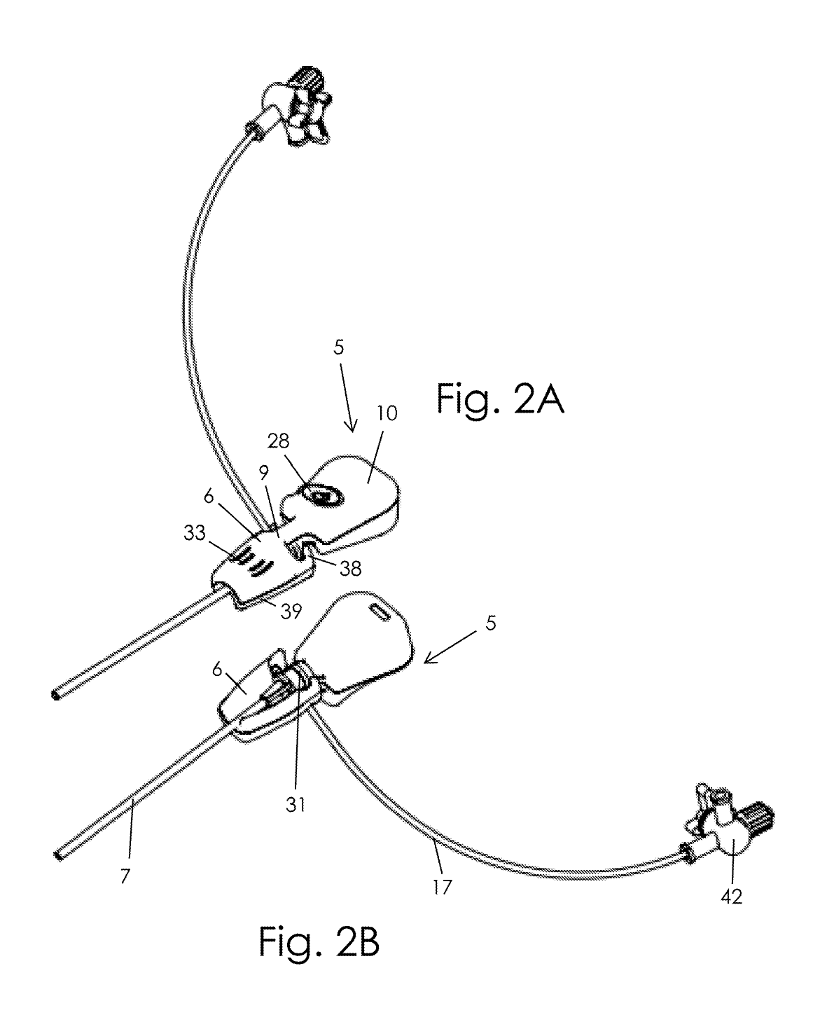

FIG. 3 illustrates a remote control system 20 by which RCU 15 communicates with MPU 25 by infrared (IR) signals.

RCU 15 comprises a Light Emitting Diode at the Infrared band (IR LED) 11, from which light signals propagate to MPU 25. An LED driver 19 configured with one or more elements is adapted to transmit digital data generated by encoder 12 or microcontroller 13 by alternating power to IR LED 11. The software code of microcontroller 13 may control encoder 12. Battery 16 supplies electrical energy to encoder 12 and/or microcontroller 13 and LED driver 19. An activation button 17, when pressed, connects battery 16 to the electrical circuit.

MPU 25 comprises a photodiode 26, which may be covered with a filter to filter out non-IR light, for converting the received IR light to electrical analog signals, a digitizer 27 for converting the analog signals to digital signals, and a decoder 29 for translating the digital signals received from digitizer 27 to a control signal. Motor driver 22 in turn transmits the control signal to pump motor 23. Switch 24 connects motor driver 22 to a manually pressable button 26 or to the other components of the electrical circuit.

Battery 27 supplies electrical energy to decoder 29, microcontroller 21 provided with software code for controlling pump motor 23, digitizer 27 and motor driver 22. Alternatively, microcontroller 21 is able to also generate the control signal without need of decoder 29. If so desired, decoder 29 is able to send operative commands to microcontroller 21 in order to perform various operations.

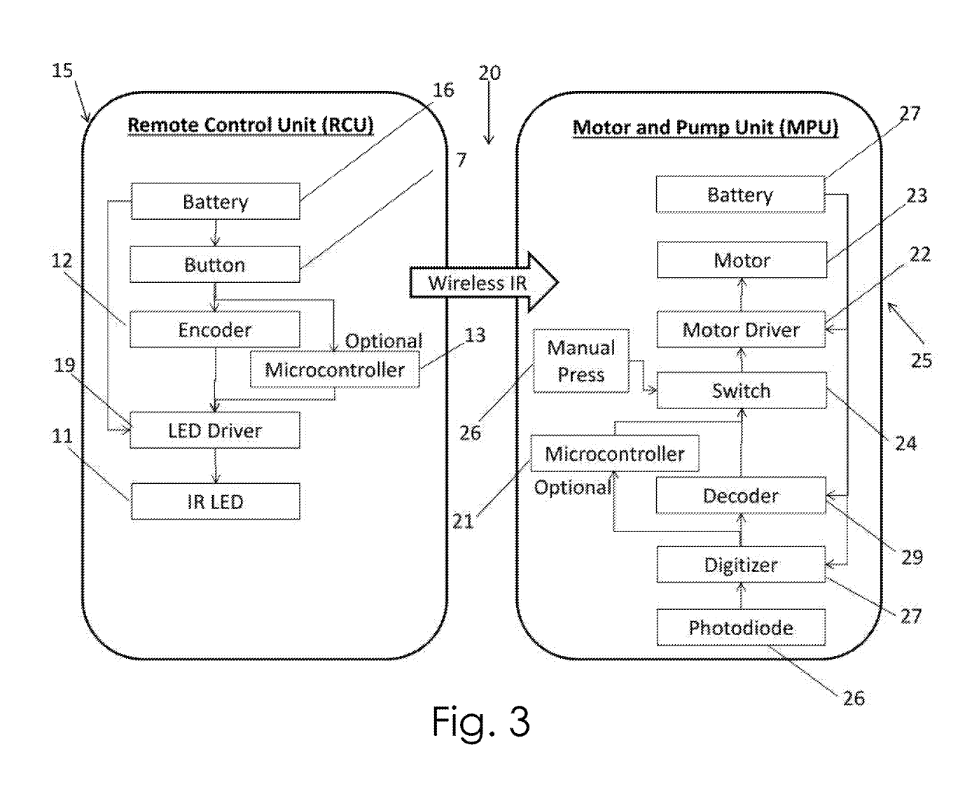

FIG. 4 illustrates a remote control system 30 by which RCU 35 communicates with MPU 45 by unidirectional radio frequency (RF) signals, generally in the industrial, scientific and medical (ISM) frequency bands ranging from 433.92 MHz to 2.4 GHz so as not to disrupt normal radio communication.

RCU 35 is identical to RCU 15 of FIG. 3, with the exception of electronic RF transmitter module 36 for converting analog or digital signals to RF signals by high frequency modulation and analog conditioning circuitry 37 to effectively propagate the RF signal with the use of operational amplifiers and an antenna. Several modulation methods may be implemented, such as amplitude modulation (AM), frequency modulation (FM,) phase modulation (PM), amplitude-shift keying (ASK), amplitude and phase-shift keying (APSK), frequency-shift keying (FSK), multiple frequency-shift keying (MFSK), minimum-shift keying (MSK), Gaussian minimum shift keying (GMSK), phase-shift keying (PSK), and quadrature-phase shift keying (QPSK).

MPU 45 is identical to MPU 25 of FIG. 3, with the exception of analog signal conditioning circuitry 47 using low noise amplifiers, filters and an antenna to selectively distinguish the RF signal, and the electronic RF receiver module 49 for converting the RF signal to an analog or digital signal by reduction of high frequency modulation to a slower signal.

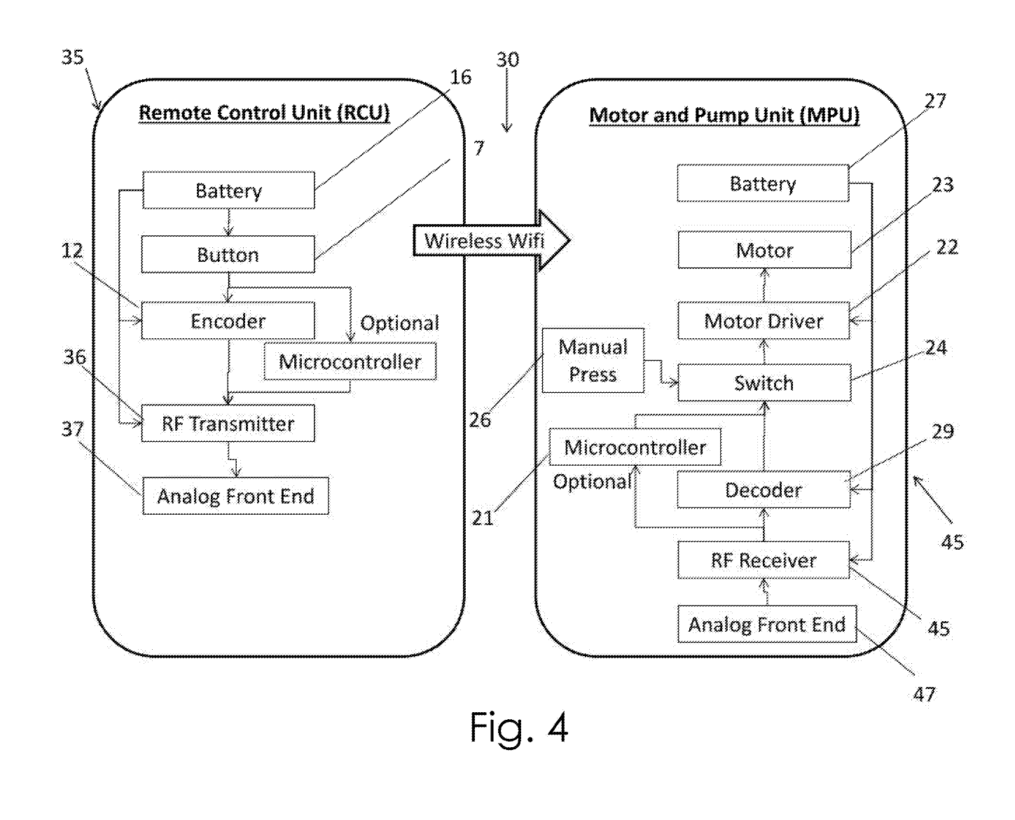

FIG. 5 illustrates a remote control system 50 by which RCU 55 communicates with MPU 60 by bidirectional radio frequency (RF) signals, generally in the ISM frequency bands ranging from 433.92 MHz to 2.4 GHz so as not to disrupt normal radio communication.

RCU 55 is identical to RCU 35 of FIG. 4, with the exception of modem 57, which may be a hardware modem or a software modem, for converting digital signals to analog signals and vice versa. The signal in communication with modem 57 is fed to or from RF transceiver 36.

MPU 60 is identical to MPU 45 of FIG. 4, with the exception of modem 63, which may be a hardware modem or a software modem, for converting digital signals to analog signals and vice versa. The signal in communication with modem 63 is fed to or from RF transceiver 49.

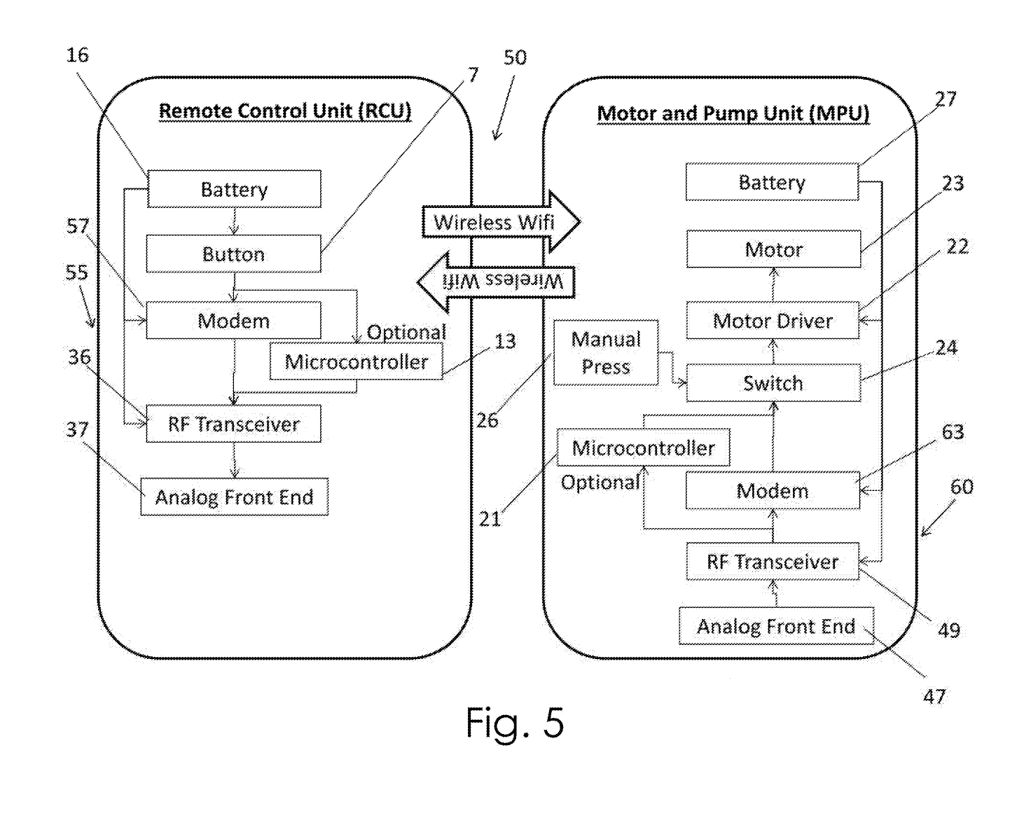

FIG. 6 illustrates a remote control system 70 by which RCU 75 communicates with MPU 80 by high frequency RF signals operating according to various protocols such as ISM 2.4 GHz Bluetooth, ISM 2.4 GHz Zigbee, ISM 2.4 GHz Wi-Fi and 60 GHz WiGig.

In this embodiment, a microprocessor 77 interfaces with a networking protocol stack 79 in order to generate commands for transmitting signals over the corresponding network via RF transceiver 36. Protocol stack 79, which may be a:hardware or software module, includes the functionality of various components such as a coder, encoder, encryption, decryption, modulation, demodulation, formatting, and timing in accordance with the given protocol standard. In order to achieve the high performance needed to communicate with protocol stack 79, microprocessor 77 comprises a Direct Memory Access (DMA) and Memory Management Unit (MMU) for providing deep memory stack operations, large vector computations and manipulations, multitasking and time slotting. MPU 80 in turn has its own microprocessor 82 and networking protocol stack 84 with which it interfaces, after receiving the transmitted signals via RF transceiver 49.

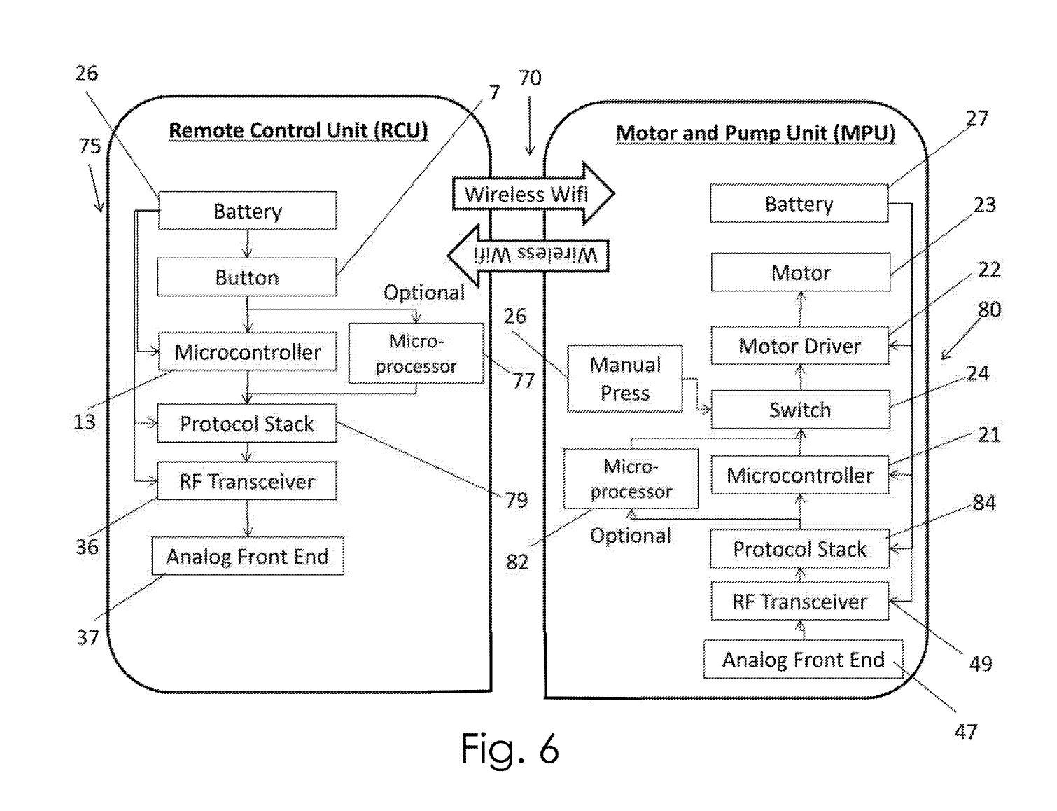

FIG. 7 illustrates a remote control system 90 by which RCU 95 is embodied by a passive tag, i.e. one lacking its own power supply, and returns RF signals to MPU 105, which functions as an active reader or interrogator.

MPU 105 is similar to MPU 80 of FIG. 6, while active reader circuit 106 comprising microcontroller 21, protocol stack 84 and RF transceiver 49 generates an RF interrogating signal and transmits the same to RF transponder 101 of RCU 95, waiting to read its response.

RCU 95 comprises analog signal conditioning circuitry 97 which comprises a bi-directional antenna 98, preferably in the form of a coil of wire, and a capacitor 99 for cooperating with the coil inductance to constitute a tuned circuit that resonates at the frequency of the interrogating signal. The RF signal is picked up by antenna 98, which collects its energy in order to power transponder 101 and retransmit the received RF signal.

Once transponder 101 is energized, it responds to the interrogating signal with an embedded code accessed from protocol stack 103. The embedded code corresponds to the format and timing of protocol stack 103, and its content may be a batch identification number, a singular identification number, a rolling code number, and encrypted data.

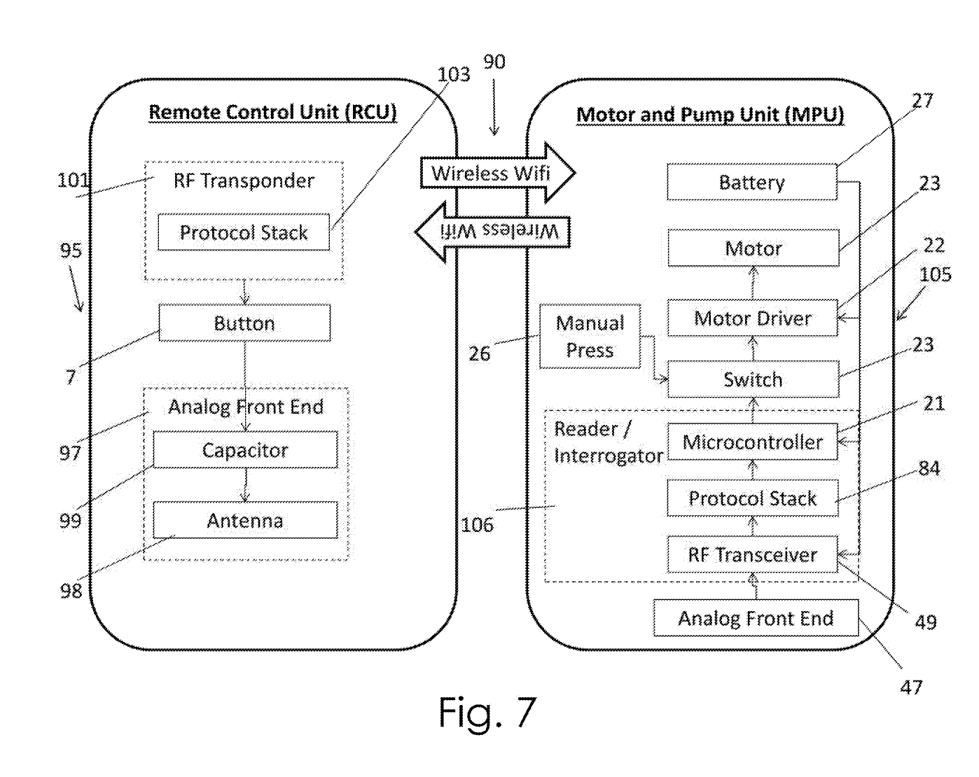

FIG. 8 illustrates a remote control system 110 by which RCU 115 communicates with MPU 125 by audio frequency (AF) signals, usually in the form of ultrasound (US) signals not heard by the human ear.

RCU 115 is identical to RCU 35 of FIG. 4, with the exception of AF transmitter 116 for generating analog AF signals and analog front end 117, e.g. a buzzer, speaker or transducer, for converting the electrical AF signals to audible signals by interaction with phonons, or energy bundles of vibrational energy.

MPU 125 is identical to MPU 45 of FIG. 4, with the exception of analog front end 127, e.g. a microphone, for capturing the transmitted audible signals and converting them to analog electrical signals, and AF receiver 129.

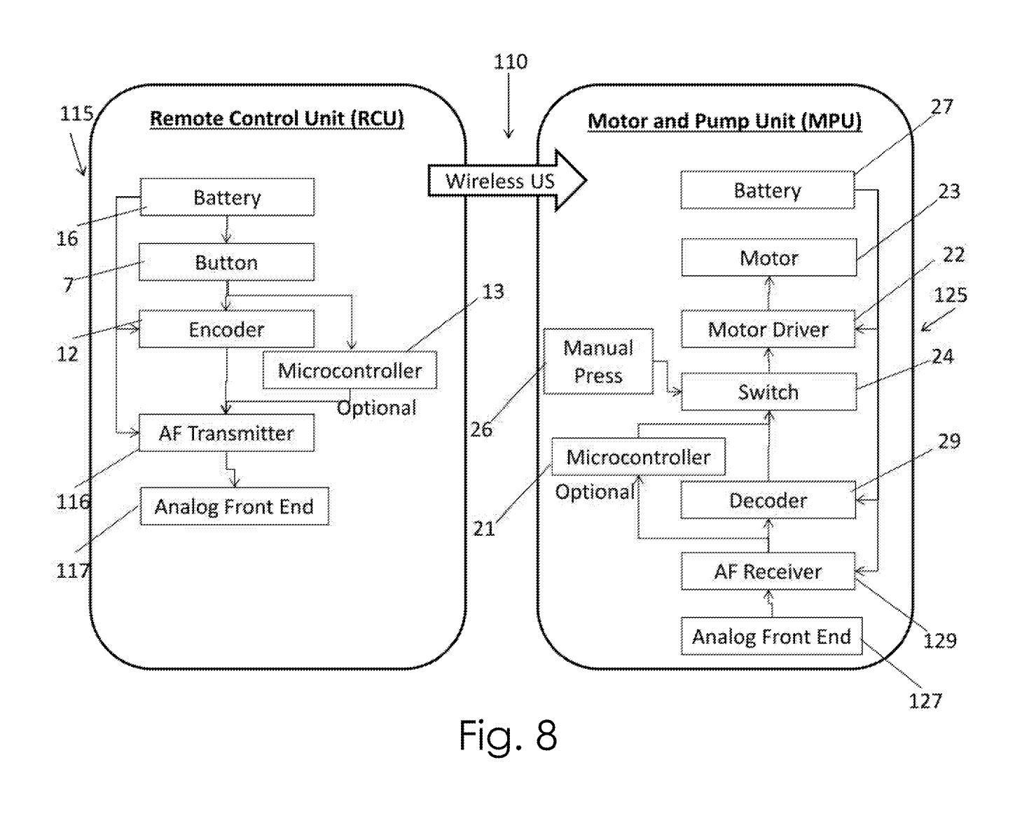

FIG. 9 illustrates another embodiment wherein the RCU is configured by a ring 135 worn on the finger of the physician performing a catheterization procedure. A schematically illustrated PCB 138 for providing the electronic capabilities of RCU 135 according to any of the embodiments described above is embedded within the ring, and is connected to finger depressable activation switch 132 and safety switch 139 housed within the ring.

In another embodiment, the remote control unit may be connected to the actuator by a wired connection through which the activation signal is transmittable.

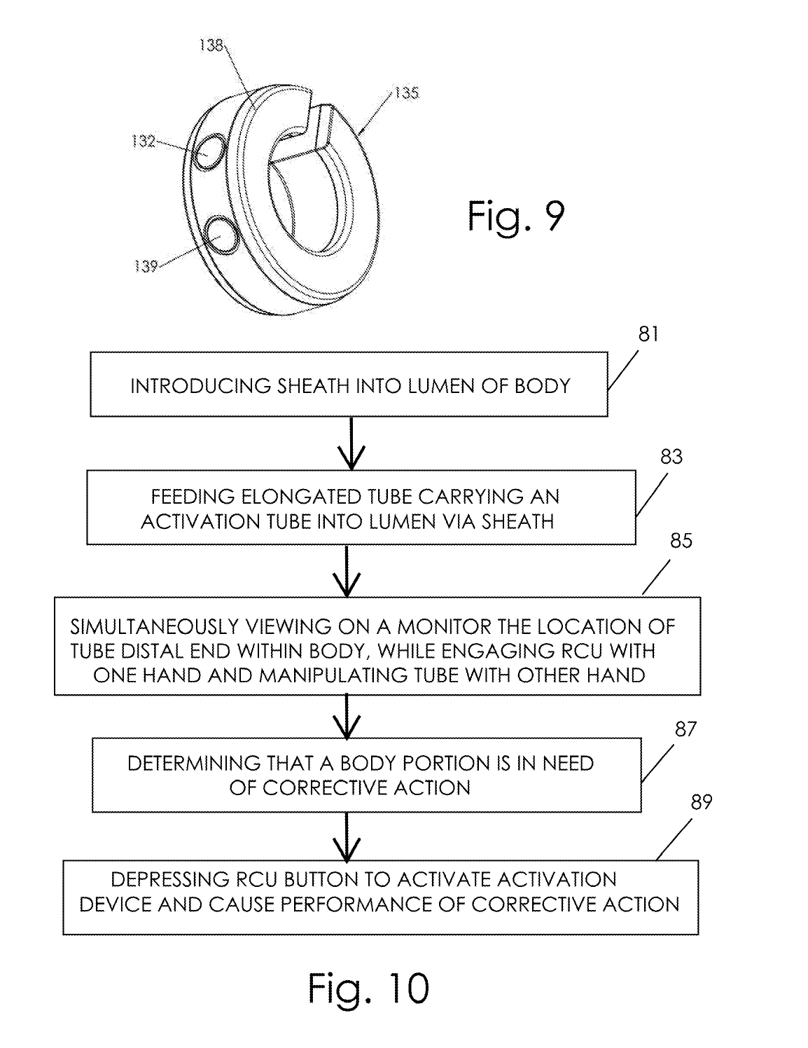

In use, as illustrated in FIG. 10, a physician or any other operator first introduces a sheath, such as an introducer sheath or a trocar, into the lumen of a body in step 81 and then feeds a catheter or any other elongated tube carrying an activation device at its distal end into the lumen via the sheath in step 83 to initiate an intraluminal operation, such as within a blood vessel, urinary bladder or a blocked pipe. By virtue of the unique configuration of the remote control system according to any embodiment described hereinabove, the operator is able to simultaneously view on a monitor in step 85 a body portion corresponding to an instantaneous location of the tube distal end within the body, while engaging the RCU with one hand and manipulating the tube with the other hand. Manipulation of the tube is facilitated by the reactive force applied onto the RCU shell by a finger of the operator. The body portion is visualized by various imaging means such as fluoroscopy and ultrasound imaging. In contrast to prior art methods whereby the operator periodically loses eye contact with the monitor in order to operate the actuator or request assistance from a practitioner, an operator using the remote control system of the present invention is able to continuously view the body portion corresponding to an instantaneous location of the tube distal end within the body throughout the intraluminal operation.

During the intraluminal operation, the operator receives a tactile sensation which is indicative of a degree of body related resistance to displacement of the tube. The operator accordingly is able to determine in step 87 whether the body is in need of a corrective action in response to one or both of the tactile sensation and viewing. Upon determining that the body portion is in need of a corrective action, one of the operator's fingers is moved to the RCU housing in order to depress a button in step 89 for actuating the activation device and for thereby performing the corrective action, while at least another finger remains on the shell.

The operator continues to view the body portion on the monitor following the corrective action. In no addition actions need to be taken, the activation device is deactivated, for example by depressing the RCU housing button once again, and retracting the activation device by suitably manipulating the tube.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1. A remote control system for use in intraluminally or intravascularly located operations, comprising:
a) an activation device located near or at a distal end of an invasive element, for performing a desired intraluminally or intravascularly located operation;
b) an actuator for said activation device near or at a proximal end of said invasive element; and
c) a remote control unit (RCU) positioned within transmission range of said actuator, wherein said RCU has a communication device for transmitting an activation signal for initiating operation of said activation device to said actuator;
wherein the RCE is attached to an invasive introducer device comprising a percutanteously insertable tube through which the invasive element is introducible into a bodily part.

2. The control system according to claim 1, wherein the invasive element is a catheter or a trocar.

3. The control system according to claim 1, wherein the introducer device is an introducer sheath having a side arm tube through which medicament or blood is injectable.

4. The control system according to claim 3, wherein the RCU comprises a hollow shell for securing and receiving the introducer sheath within its interior, and a closed housing within which is retained a printed circuit board (PCB) for providing electronic capabilities of the RCU.

5. The control system according to claim 4, wherein an activation switch for initiating operation of the actuator is connected to the PCB and protrudes from the housing.

6. The control system according to claim 4, wherein the shell and the housing are separated by a spacer extending therebetween, a protrusion by which the side arm tube of the introducer sheath is fixatable extending from a circumferential edge of the shell towards said spacer.

7. The control system according to claim 6, wherein the RCU is of such sufficiently light weight that a reactive force is appliable to the shell by a finger during a catheterization procedure.

8. The control system according to claim 7, wherein the spacer is sufficiently short to facilitate displacement of a second finger from the shell to the housing in order to depress the activation switch while a first finger remains in engagement with the shell, during the catheterization procedure.

9. (canceled)

10. The control system according to claim 1, wherein the activation signal is transmittable to the actuator in response to interaction with the RCU by no more than three fingers.

11. (canceled)

12. The control system according to claim 1, wherein the activation signal is selected from the group consisting of:
a) a wirelessly transmittable signal;
b) an encoded signal;
c) an infrared signal;
d) a radio frequency signal;
e) an audio frequency signal; and
f) an unidirectional or bidirectional radio frequency signal in the ISM frequency bands.

13-16. (canceled)

17. The control system according to claim 12, wherein the RCU comprises a microprocessor for interfacing with a networking protocol stack in order to generate the activation signal.

18. The control system according to claim 17, wherein the RCU is a passive tag which is operable to respond to an interrogating signal generated by the activation device.

19. (canceled)

20. The control system according to claim 1, wherein the actuator is a mechanical member or an electrical member powered by an AC or DC current source, and activated by the RCU.

21. The control system according to claim 20, wherein the actuator is a Motor and Pump Unit (MPU) for draining intraluminally accumulated liquid by applying subatmospheric pressure.

22. The control system according to claim 1, wherein the activation device is an atherectomy device for removing atheromatous material from the walls of a blood vessel.

23. The control system according to claim 4, wherein a safety switch for preventing inadvertent initiation of the actuator is connected to the PCB and protrudes from the housing, initiation of the actuator being enabled only if said safety switch is depressed within a predetermined time after the activation switch has been depressed.

24. The control system according to claim 23, further comprising a visual or audible indicator for indicating actuator initiation.

25. (canceled)

26. The control system according to claim 1, wherein the RCU and actuator are spaced by a distance ranging from 1 to 2000 cm that is variable during the course of the operation.

27. A method for remotely controlling an activation device, comprising the steps of:
a) introducing a tubular sheath into a lumen of a body, wherein a remote control device is attached to a portion of said sheath externally to said body;
b) feeding a flexible and elongated tube into said lumen via said sheath to initiate an intraluminal operation, wherein an activation device is affixed to a distal end of said tube;
c) by an operator, while displacing said tube within said lumen with one hand to receive a tactile sensation which is indicative of a degree of body related resistance to displacement of said tube and engaging said remote control device with another hand, simultaneously viewing on a monitor a portion of said body corresponding to an instantaneous location of said tube distal end; and
d) after determining that said body portion is in need of a corrective action in response to one or both of said tactile sensation and said viewing, depressing at least one button of said remote control device to actuate said activation device and to thereby perform said corrective action.

28. The method according to claim 27, wherein the operator continuously views on the monitor one or more portions of the body corresponding to an instantaneous location of the tube distal end within the body throughout the intraluminal operation.
